(19) United States
(12) Patent Application Publication
(10) Pub. No.: US 2005/0063928 A1
(43) Pub. Date: Mar. 24, 2005

(54) Compositions comprising odor neutralizing metal oxide silicates

(76) Inventors: Michael C. Withiam, Landenberg, PA (US); Donald P. Conley, Conowingo, MD (US); Michael Simone, North East, MD (US)

Correspondence Address: Carlos Nieves, Esq.;J. M. Huber Corporation, 333 Thornall Street, Edison, NJ, 08837-2220, US

(21) Appl. No.: 10/666,386

(22) Filed: Sep. 18, 2003

(51) Int. Cl. A61K 7/32; A61K 7/36

(52) U.S. Cl. 424/65; 424/67

(57) ABSTRACT

Disclosed is a fluid personal care composition comprising a metal oxide silicate and a vehicle, wherein the metal oxide silicate is capable of absorbing a malodorous compound. These fluid personal care compositions that provide effective, long-lasting suppression of the malodors associated with human perspiration. The personal care composition may be in the form of a solid stick deodorants, liquid roll-on deodorants, aerosol, and pump spray deodorants, semi-solid gel deodorants, soap bars, and deodorant lotions and creams.

### BACKGROUND OF THE INVENTION

A broad array of topical personal care and personal hygiene products are available for application to human skin to counteract malodors associated with the human body, particularly those malodors resulting from and associated with perspiration. These products include deodorants, antiperspirants, foot and body powders, body sprays, and especially sports and athletic sprays and powders.

Conventional personal care and cosmetic products may be formulated to counteract and neutralize body malodors in a variety of ways. For instances, these malodors may be “masked” or concealed by placing a sufficient amount of perfume composition in the deodorant in order to hide or cover the malodor. Perfumes provide the additional benefit of imparting a desirable fragrance, such as a variety of different fresh, pastoral, or musk scents, to a cosmetic or personal care product. However, “masking” also has distinct limitations. Some malodors cannot be masked simply by adding perfumes, because they are highly volatile (and therefore diffuse quickly into the air) or because they are extremely potent. Indeed, in some cases it may be impossible to add sufficient amounts of perfume in order to sufficiently conceal the underlying malodor without also giving the personal care product an overly strong, perfumed odor.

Another way of counteracting body malodor is through the use of topical antimicrobials such as triclosan. Perspiration-associated body malodors are typically the result of interaction between microbes, perspiration and triglyceride secretions from the sebaceous glands, which combine to produce malodorous and pungent fatty acids. Thus, by controlling the microbe population on the skin's surface, the malodor can be eliminated or reduced in intensity.

However, the use of antimicrobial agents also presents certain problems. Overuse of antimicrobial agents is strongly discouraged because it can potentially contribute to the development of disease-resistant microbes, and additionally the build-up of antimicrobial agents in the human body may have unknown side effects. Moreover, adding these antimicrobials to a typical deodorant composition may cause the deodorant to irritate the skin.

Another approach that avoids the aforementioned problems while also reducing malodor involves the use of odor absorbers, such as activated charcoal and zeolites. These odor absorbing compounds function by absorbing odors and perspiration, and unlike the aforementioned treatment compounds they do not irritate the skin or impart an overly perfumed scent to the composition. However, charcoal and zeolite odor absorbers have the disadvantage that as they get wet (e.g., they come into contact with perspiration) they become ineffective at odor absorption. For similar reasons, these odor absorbers can also be difficult to formulate into compositions that contain even small quantities of water.

Given the forgoing there is a continuing need for cosmetic and personal care products that contain an ingredient to effectively suppress the malodors associated with human perspiration. Such a malodor-suppressing ingredient preferably has no other health or hygienic side effects, and can be easily formulated into a wide variety of cosmetic and personal care products.

### BRIEF SUMMARY OF THE INVENTION

The invention includes a fluid personal care composition comprising a metal oxide silicate capable of absorbing a malodorous compound and a vehicle; the metal oxide silicate being of the formula: x MO:SiO₂, wherein x is M is a multivalent metal, and x is the number of moles of metal oxide, x being greater than 1; and the metal oxide silicate has an oil absorption of greater than 50 ml/100 g.

The invention also includes a method of inhibiting body odor by applying to the skin an effective amount of a personal care composition comprising a metal oxide silicate capable of absorbing a malodorous compound, the metal oxide silicate being characterized by the formula: x MO:SiO₂, wherein x is M is a multivalent metal, and x is the number of moles of metal oxide, x being greater than 1; and the metal oxide silicate has an oil absorption of greater than 50 ml/100 g.

### DETAILED DESCRIPTION OF THE INVENTION

All parts, percentages and ratios used herein are expressed by weight unless otherwise specified. All documents cited herein are incorporated by reference.

By “fluid personal care compositions” it is meant compositions that contain greater than 20% of one or more ingredients acceptable for use in cosmetics that are liquid at temperatures less than 100° C. and greater than 32° C., such as deodorants, anti-perspirants, athletic sprays, body sprays, hair conditioners, shampoo, skin conditioners, body washes, liquid bath soaps, facial cleansers, make-up removers, baby baths, hand soaps and the like.

The present invention includes topical fluid personal care compositions comprising an odor-absorbing, odor-neutralizing metal silicate along with at least one acceptable vehicle (such as diluents or carriers) for the odor-absorbing metal oxide silicate, so as to facilitate the metal oxide silicate's distribution when the composition is applied to the skin. The invention also includes the fluidization and application from a volatile vehicle as in the case of aerosols. (Suitable vehicles, as well as other suitable personal care composition ingredients are discussed in greater detail, below). The silicates act as odor absorbents and neutralizers to absorb and neutralize body malodors, particularly body malodors associated with perspiration. By incorporating these metal oxide silicates, a wide variety of personal care compositions may be produced that provide effective, long-lasting absorption and neutralization of odors. This allows effective body malodor suppression without the overuse of perfumes or reliance on antimicrobial agents. In addition to these benefits, the metal oxide silicates also improve the “feel” of personal care compositions in which it is incorporated. Particularly, the personal care compositions have a smoother, drier feel when applied and in contact with the skin.

That fluid personal care compositions incorporating metal oxide silicate are capable of providing effective odor neutralization and suppression would itself be surprising to a person of ordinary skill in the art. This is because the particulate metal oxide silicates tend to be intermixed and coated with several other ingredients, and thus would seem incapable of neutralizing and suppressing body malodors. However, by the present invention fluid personal care compositions have been formulated that fully incorporate metal oxide silicate particles without diminishing the ability of the silicate particles to absorb and neutralize odors.

While not wishing to be limited by theory, it is believed that the metal oxide silicates in the cosmetic compositions prepared according to the present invention absorb both malodors originating from human skin as well as absorb the fatty acids found on the skin. Thus, these metal oxide silicates are believed to offer two measures to neutralize body malodors: they not only absorb the malodors themselves, but they also reduce the quantities of fatty acids that are part of the cause of the malodors. The high oil absorption capacity of many of the metal oxide silicates encourage the movement of the malodorous compounds into the intraparticle pores and interstices that are formed within the metal oxide silicates.

While it is conceded that several naturally occurring metal-oxide containing substances would offer some noticeable amount of odor reduction, generally synthesized materials are preferred. Synthesized materials, such as silicates of the present invention, are preferred because they can be prepared to have higher absorption and odor neutralization than naturally occurring substances. Nonetheless, mineral silicates like Bredigte, Diopside, Enstatite, calcium silicate hydrate (both Type I and Type II), Tobermorite, Okenite or other natural materials would be useful in the application of odor reduction.

These excellent absorption and neutralization properties are themselves believed to result from the high surface area and oil absorption properties of the metal oxide silicates. The high surface area of the metal oxide silicate particles allows the chemical substances that will volatilize to form malodors and fatty acids to be easily exposed to the surfaces of the silicate particles, while the high oil absorption capacity of the calcium silicates encourages the movement of volatilized malodors and fatty acids from the particle surface and into the interior of the metal oxide silicate particles.

Thus, the present fluid personal care compositions contain synthetic amorphous metal oxide silicates that absorb volatile malodor-causing chemicals and fatty acids and thereby neutralize the malodors associated with human perspiration. Preferably the oil absorption of these silicates is between about 20 ml/100 g and about 500 ml/100 g, preferably between 50 ml/100 g and 250 ml/100 g. The surface area (BET) is preferably between about 5 m²/g to 200 m²/g, preferably between about 50 m²/g and about 200 m²/g. The pH is preferably between about 7 and 12, more preferably between about 9 and 10. The particle size is preferably less than 30 microns and more preferably less than 15 microns.

These metal oxide silicates are most typically prepared by the reaction of a reactive silica with an alkaline earth metal or transition metal reactant, preferably an alkaline earth metal oxide or hydroxide, or mixtures of multiple metal oxides or hydroxides. Because the final properties of the silicate are dependent on the reactivity of the silica, the silica source is preferred to be the reaction product of a soluble silicate, such as, but not limited to sodium silicate, and a mineral acid, such as sulfuric acid.

While it has been shown that single metal oxide silicates are useful and provide benefits reducing malodors, the present invention teaches that certain combinations offer still further benefits. Combinations of metal oxides that are each capable of delivering a benefit can be prepared by co-reacting different metal oxide reactants with reactive silica to form a complex amorphous silicate with enhanced benefit over the individual metal oxide silicate or over the simple blend of the components. The complex silicates offer more effective and more dermatologically acceptable materials for odor control.

Normal skin generally has a pH of 5.4-6.2. Application of cosmetics having a pH well outside of the pH range of normal skin can aggravate the skin, which can lead to changes in skin pH and surface chemistry and result in irritation. The present invention discloses processes for making absorbent metal oxide silicates, with these silicates being both useful in reducing body malodor, while having a lower pH so as to be less aggravating to the skin surface. The discovery of such processes is based upon the well-accepted fact that various metal oxides have different degrees of solubility and inherent alkalinity, and through manipulation of the ratios of the oxide components in the amorphous silicate, a silicate is produced that has a unique combination of dermatologically-acceptable pH without compromising its odor reduction capacity.

Specifically, these efficacious metal oxide silicates having dermatologically-acceptable pH are preferably manufactured by the synthesis of the silicates while the silicates suspended in their reaction medium. While this is the preferred method, the methods of the present invention are not limited to the direct treatment of the metal oxide silicate in dry particulate form. Adjustment of pH can be made using dilute mineral acids or organic acids. Preferably the acid is one that is well known to be acceptable in cosmetics formulations, such as citric or carbonic acid. Most preferably the acid is carbonic acid, added in either liquid or gaseous form to provide the silicate absorbent with the desired dermatologically acceptable pH.

Fluid personal care compositions prepared according to the present invention comprise about 0.5 wt % to about 20 wt %, preferably about 0.2 wt % to about 5 wt % of the odor neutralizing metal oxide silicate. In addition to the metal oxide silicate, the present fluid personal care compositions will also comprise one or more dermatologically acceptable cosmetic ingredients.

Dermatologically acceptable cosmetic ingredients include first and most importantly a diluent or carrier. The vehicle, diluent or carrier may be selected from a wide range of ingredients. The vehicle may comprise water and/or a water-miscible or dispersible organic liquid or liquids and alternatively or additionally a water-immiscible liquid or liquids and waxes. The cosmetically acceptable vehicle will preferably form from 95% to 99.2% by weight of the composition, and can, in the absence of other cosmetic adjuncts, form the balance of the composition. The vehicle may be aqueous, non-aqueous or a combination of both, such as an emulsion. In a combination vehicle, an oil or oily material may be present, together with one or more emulsifiers to provide either a water-in-oil emulsion or an oil-in-water emulsion, depending largely on the average hydrophilic-lipophilic balance (HLB) of the emulsifiers employed. This also includes multiple emulsions: water-in-oil-in-water or an oil-in-water-in- oil emulsions.

In the case where the composition contains a combination of aqueous and non-aqueous vehicle components, the aqueous phase can be from about 90 wt % to about 10 wt % of the vehicle, as can the non-aqueous phase. In an embodiment of the invention where the vehicle is aqueous or is comprised of a mixture of aqueous and non-aqueous components, preferably the vehicle is at least 80 wt % water, by weight of the vehicle. Preferably, water comprises at least 85 wt % of the inventive composition, and most preferably from 90 to 95 wt % of the composition.

In an embodiment of the invention where the vehicle is comprised of non-aqueous components, the dermatologically acceptable non-aqueous cosmetic ingredients in the vehicle will usually form from 80% to 99.9% by weight of the composition, and may, in the absence of other cosmetic adjuncts, form the balance of the composition.

Examples of suitable non-aqueous carriers may include alcohols, polyalkoxylated glycols (such as propylene glycol), volatile and nonvolatile liquid silicone carriers (such as cyclicsilicone polymers), hydrocarbon and mineral oils and branched chain hydrocarbons And combinations of hydrocarbons useful as propellants. Specific, non-limiting examples of organic liquids suitable for use include octyldodecanol, butyl stearate, diisopropyl malate, and combinations thereof. Also suitable for use are acrylic acid-based polymers.

It is desirable that the odor absorbing ingredient in the inventive compositions remains substantially localized in the region of the body to which it has been topically applied. In order to assist this to happen and also to enable alternative dispensers for the composition to be employed, the vehicle may be thickened or structured, for example by introducing one or more materials for that purpose. Thickened or structured compositions commonly adopt the form of firm sticks, soft solids and creams. In such circumstances, the materials are often referred to as structurants or gellants and may sometimes alternatively be called thickeners, depending on the final form of the composition. The vehicle may be further diluted with a volatile propellant and used as an aerosol; may be mixed with an additional liquid and/or other ingredients and used, for example, as a roll-on or squeeze-spray product; or mixed with one or more thickeners and/or structurants and used, for example, as a gel, soft solid, or solid stick product.

Exemplary thickeners are cross-linked polyacrylate materials available under the trademark Carbopol from the B.F. Goodrich Company. Gums may be employed such as xanthan, carrageenan, gelatin, karaya, pectin and locust beans gum. Under certain circumstances, the thickening function may be accomplished by a material also serving as a carrier or emollient vehicle. For instance, silicone gums in excess of 10 centistokes and esters such as glycerol stearate have such dual functionality. A thickener will usually be present in amounts anywhere from 0.1 to 20% by weight, preferably from about 0.5% to 10% by weight of the composition.

Other dermatologically acceptable cosmetic ingredients include rheology affecting agents (i.e., rheology modifiers) such as solidifying agents and gellants. The solidifying agents act to provide solidity to a personal care composition so that they are in solid (or semi-solid) form at room temperature. Suitable solidifying agents include especially high melting point waxes (melting points between 65° C.-110° C.) which include hydrogenated castor oil, paraffin, synthetic wax, ceresin, beeswax, and other such waxes. Also acceptable are low melting point waxes (melting points between 37° C.-65° C.), which include fatty alcohols, fatty acids, fatty acids esters, fatty acid amides, and the like.

Gellants are used in the case of solid stick compositions, to give the stick an appropriate consistency and provide an appropriate gel matrix and product hardness at the completion of processing. The gelling agents will vary depending on the particular form of the personal care composition and whether the personal care composition is aqueous or nonaqueous. Suitable gellants include esters and amides of fatty acid or hydroxy fatty acid gallants, fatty acid gellants, salts of fatty acids, esters and amides of fatty acid or hydroxy fatty acid gellants, cholesterolic materials, lanolinolic materials, fatty alcohols, triglycerides, and other suitable solid, non-polymeric gellants. Preferred gellants (for both aqueous and nonaqueous compositions) include fatty alcohols, most preferably stearyl alcohol. Amounts of these gellant components may range anywhere from 0.001% up to 20% by weight of the composition.

The inventive compositions may contain any of a number of desired “active” ingredients, including drug substances such as anti-inflammatory agents, topical anesthetics, antimycotics, etc.; skin protectants or conditioners; humectants; and the like, depending on the intended uses for the formulations.

The fluid personal care products prepared according to the present invention may also include other optional components. The CTFA Cosmetic Ingredient Handbook, Eighth Edition, 2000, which is incorporated by reference herein in its entirety, describes a wide variety of cosmetic and pharmaceutical ingredients commonly used in skin care compositions, and which are suitable for use in the compositions of the present invention. These optional components include pH buffering agents, additional malodor control agents, fragrance materials, dyes, and pigments, preservatives, skin aids (e.g., aloe), cosmetic astringents, liquid or solid emollients, emulsifiers, film formers, propellants, skin-conditioning agents, such as humectants, skin protectants, solvents, solubilizing agents, suspending agents, surfactants, waterproofing agents, viscosity increasing agents (aqueous and nonaqueous), waxes, wetting agents, and other optional components. Amounts of these adjunct components may range anywhere form 0.001% up to 20% by weight of the composition.

The products themselves may be formulated to be in a variety of forms, such as solid and semi-solid stick deodorants (such as emulsion sticks or suspensoid sticks), roll-on deodorants, and deodorant aerosol and pump-sprays, and even soap bars.

The fluid personal care compositions of the present invention may be prepared by any known or otherwise effective technique suitable for providing a fluid personal care composition having the essential materials described herein. Techniques for forming such compositions are very well known in the art. The present invention is not dependent upon any particular formulation technique, it being recognized that the choice of specific formulation components may well make necessary some specific formulation procedure.

Methods for preparing the fluid personal care compositions of the present invention include conventional formulation and mixing techniques. Many variations of formulating the compositions of the present invention exist, and all are considered within the scope of the present invention. Suitable methods include combining the metal oxide silicate odor absorbing/neutralizing agent with part or all of the liquid vehicle. A liquid may be entirely absorbed into the metal oxide silicate, and if so, additional liquid or liquids and other materials are added until the metal oxide silicate is evenly dispersed. A thickener or gellant is added and the composition is mixed and may be heated, if required for homogenous incorporation. Adjunct and/or additional materials may be added at this point, and the batch may be allowed to cool, if necessary. The thickened or gelled composition is allowed to viscosity or solidify in a suitable container or dispenser.

The invention will now be described in more detail with respect to the following, specific, non-limiting examples.

### EXAMPLES 1-4

In these examples, calcium silicates with mole ratios varying from 1:1 to 3:1 of calcium oxide to silica were produced.

In a first step of these examples, amorphous silica suitable for use in the production of the inventive metal silicates was prepared by adding sulfuric acid to a dilute waterglass solution in a well-agitated mixing vessel to affect the precipitation of amorphous hydrated silica. Specifically, a total of 1052 liters of sulfuric acid at a concentration of 11.5% was added at a rate of 17.8 lpm (liters per minute) to 1893 liters of waterglass solution (3.3 SiO₂/Na₂O mole ratio) containing 13% sodium silicate solids while mixing at a temperature of 95° C. The addition of the sulfuric acid was continued until a pH of 5.5 was obtained, and the reaction mixture was digested for 1 hr. The resulting suspension of silica particles was recovered by filtration, and washed and dried to form a finely divided reactive silica powder. It is equally useful to retain the undried material in the form of a filtered cake, as an intermediate material for subsequent synthesis.

The reactive silica produced above was then slurried in water to a specified % solids in a reaction vessel equipped with a constant torque agitator and paddle blades. Then water and lime slurry at specified % solids were added to the reactor. The reactor temperature was raised to a specified temperature and the reaction mixture digested. The resulting metal silicate was then filtered, dried and milled. Additionally, Example 2 was further dehydrated by heating the previously dried, milled material at 900° C. for 60 minutes. Example 2 converted to a crystalline form of calcium silicate on dehydration.

Reaction variables for Example 1-4 are given in Table 1 below.

TABLE 1

| | Example 1 | Example 2 | Example 3 | Example 4 |
|---|---|---|---|---|
| Reactive silica weight, | 120.3 | 187.4 | 95.3 | 0.462 |
| kg | | | | |
| Reactive silica solids, % | 22.3 | 20.34 | 17.7 | 14.6 |
| Water, kg | 540.3 | 200 | 512.1 | 0.4 |
| Lime slurry, kg | 172.1 | 254.6 | 233.8 | 1.434 |
| Lime slurry, % solids | 19.9 | 18.0 | 18.7 | 16.2 |
| Reaction temperature, | 95 | 60 | 95 | 95 |
| ° C. | | | | |
| Digestion time, min. | 60 | 30 | 60 | 60 |
| Dehydration temperature, | — | 900 | — | — |
| ° C. | | | | |
| Dehydration time, min. | 0 | 60 | 0 | 0 |

Examples 1-4 properties are summarized in Table 4 below.

### EXAMPLES 5-8

In these examples, the pH of the calcium silicate as produced in example 1, 3 and 4 was lowered to a more cosmetically acceptable pH by treating the material either before or after drying with a cosmetically acceptable acidic moiety.

In Examples 5 and 6, a quantity of calcium silicate produced in Example 1 was heated to 50° C. and treated with carbon dioxide gas or 5% citric acid solution, respectively, to a pH of 8.9 and then digested, filtered, dried and milled.

In Example 7, the product of Example 3 was reproduced, except after the 60 minute digestion time at 95° C., CO₂ gas was bubbled through the slurry for 104.5 minutes at a rate of 84.95 lpm until the slurry pH reached 8.7. The slurry was then digested for 15 minutes, filtered, dried and milled.

In Example 8, a quantity of the product of Example 4 was recovered after the 60 minute digestion before drying. Carbon dioxide gas was then bubbled though the recovered slurry, which had been heated to 50° C., for 15 minutes until the slurry reached pH 8.7 and thereafter the slurry was digested, filtered, dried and milled.

Process conditions for Example 5-8 are given in Table 2 below.

TABLE 2

| | Example 5 | Example 6 | Example 7 | Example 8 |
|---|---|---|---|---|
| Example 1, g | 320 | 320 | — | — |
| Example 3, g | — | — | all | — |
| Example 4, g | — | — | — | 250 |
| Solids, % | 10 | 10 | — | — |
| Reaction temperature, | 50 | 50 | 95 | 50 |
| ° C. | | | | |
| CO₂ addition time, min. | 15 | — | 104.5 | 16 |
| CO₂ addition rate, 1 pm | 84.95 | — | 84.95 | 84.95 |
| 5% Citric acid addition | — | 9.5 | — | — |
| time, min. | | | | |
| Citric acid addition rate, | — | 30 | — | — |
| ml/min | | | | |
| pH | 8.9 | 8.9 | 8.7 | 8.7 |
| Digestion time, min. | 15 | 15 | 15 | 15 |

Examples 5-8 properties are summarized in Table 4 below.

### EXAMPLES 9-12

In these examples, magnesium silicate (Examples 9 & 12), calcium magnesium silicate (Examples 10 & 11) were prepared. The general process scheme described in previous examples was followed with reagent substitutions and additions given in Table 3 below. In examples 10-11, to assure complete reaction of the silica source with the magnesium source before addition of the calcium source, the reactive silica, water and magnesium hydroxide were mixed together for 5 minutes before addition of the lime slurry.

TABLE 3

| | | Example | Example | Example |
|---|---|---|---|---|
| | Example 9 | 10 | 11 | 12 |
| Reactive silica weight, | 0.4703 | 0.4703 | 114.9 | 0.4615 |
| kg | | | | |
| Reactive silica solids, % | 14.3 | 14.3 | 17.6 | 14.6 |
| Water, kg | 0.5 | 0.4 | 605.2 | 0.5 |
| Mg(OH)₂, kg | 0.1104 | 0.1104 | 52.2 | 0.221 |
| Mg(OH)₂ solids, % | 51 | 51 | 56.3 | 51 |
| Lime slurry, kg | 0 | 0.4528 | 70.4 | 0 |
| Lime slurry, % solids | — | 16.5 | 18.6 | 0 |
| Additional water, kg | — | 0.2 | — | — |
| Reaction temperature, | 90 | 90 | 95 | 90 |
| ° C. | | | | |
| Digestion time, min. | 60 | 60 | 60 | 60 |

Examples 9-12 properties are summarized in Table 4 below.

### EXAMPLE 13

In this example, a magnesium zinc silicate was produced. First, a zinc silicate wetcake was made by mixing 2021 g of sodium silicate solution at a concentration of 13.3% (3.3 SiO₂/Na₂O mole ratio) and 80 g of BASF Z-Coat ZnO powder in an agitated vessel. The mixture was heated to 90° C. To the heated suspension, 11.4% sulfuric acid was added at a rate of 35 ml/min for 25.5 minutes until the slurry reached a pH of 5.83 and then it was digested for 15 minutes. The zinc silicate wetcake was recovered by filtration and washed with hot water.

423.4 g of zinc silicate wetcake at 23.6% solids was mixed with 250 g water and then 98.0 g magnesium hydroxide at 51% solids was added. The reactants were heated to 90° C. for 1 hour with mixing. The resulting magnesium zinc silicate product was filtered, dried and milled. Example 13 properties are summarized in Table 4, below.

TABLE 4

| | | | | BET | | Mean | |
|---|---|---|---|---|---|---|---|
| | Molar Oxide | | Oil | Surface | Pore | Particle | |
| | Formula | | Absorption | Area | Volume | Size | True Density |
| | CaO:MgO:ZnO:SiO2 | 5% pH | ml/100 g | m²/g | ml/g | μm | g/cc |
| Example 1 | 1:0:0:1 | 10.8 | 224 | 168 | — | — | — |
| Example 2 | 1:0:0:1 | 9.5 | 50 | 4 | — | — | — |
| Example 3 | 2:0:0:1 | 11.8 | 239 | 154 | 3.72 | 15.4 | 2.12 |
| Example 4 | 3:0:0:1 | 11.7 | 230 | 117 | — | 15.2 | 2.27 |
| Example 5 | 1:0:0:1 | 9.6 | 130 | 164 | — | — | — |
| Example 6 | 1:0:0:1 | 9.6 | 174 | 165 | — | — | — |
| Example 7 | 2:0:0:1 | 9.6 | 76 | 111 | — | — | — |
| Example 8 | 3:0:0:1 | 9.4 | 98 | 46 | — | — | — |
| Example 9 | 0:1:0:1 | 9.1 | 145 | 42 | 2.34 | 11 | 2.28 |
| Example 10 | 1:1:0:1 | 10.5 | 120 | 29 | — | — | — |
| Example 11 | 0.5:1.5:0:1 | 9.6 | 137 | 103 | 2.28 | 2 | — |
| Example 12 | 0:2:0:1 | 8.8 | 76 | 42 | 1.81 | 7.7 | 2.23 |
| Example 13 | 0:0.7:0.2:1 | 9.7 | 72 | 23 | 1.85 | 10.4 | 2.53 |

Where mentioned in this application, the surface area was determined by the BET nitrogen absorption method of Brunaur et al., as reported in the J. Am. Chem. Soc. 60, 309 (1938). True density was determined with a helium pycnometer.

Particle size was determined using a Model LA-910 laser light scattering instrument available from Horiba Instruments, Boothwyn, Pa. A laser beam is projected through a transparent cell, which contains a stream of moving particles suspended in a liquid. Light rays, which strike the particles, are scattered through angles which are inversely proportional to their sizes. The photodetector array measures the quantity of light at several predetermined angles. Electrical signals proportional to the measured light flux values are then processed by a microcomputer system to form a multi-channel histogram of the particle size distribution.

The oil absorption shown throughout this application is measured with the rubout method. In this test, oil is mixed with a silicate and rubbed with a spatula on a smooth surface until a stiff putty-like paste is formed. By measuring the quantity of oil required to have a paste mixture, which will curl when spread out, one can calculate the oil absorption value of the silicate, the value which represents the volume of oil required per unit weight of silicate to completely saturate the silicate absorptive capacity. Calculation of the oil absorption value was done according to equation (I):

$$\begin{matrix}Oil absorption = \frac{ml oil absorbed}{weight of silicate , grams} \times 100 \\ = ml oil \text{/} 100 gram silicate\end{matrix} \qquad ( I )$$

Pore volume was determined using an Autopore II 9220 Porosimeter (Micromeritics Corporation). This instrument measures the void volume and pore size distribution of various materials. Mercury is forced into the voids as a function of pressure and the volume of mercury intruded per gram of sample is calculated at each pressure setting. Total pore volume expressed herein represents the cumulative volume of mercury intruded at pressures from vacuum to 60,000 psi. Increments in volume (ml/g) at each pressure setting are plotted against the pore radius corresponding to the pressure setting increments. The peak in the intruded volume versus pore radius curve corresponds to the mode in the pore size distribution and identifies the most common pore size in the sample.

### EXAMPLE 14

In this example, several calcium silicates made in Examples 1-8, as well as comparative substances, Hubersorb 600 available from J.M. Huber Corporation, Edison, N.J. and the mineral wollastonite, available from R.T. Vanderbilt Company, Norwalk, Conn., were evaluated in an in vitro odor absorption capacity test described below. Results of the odor capacity evaluation are summarized in Table 5.

Trans-3-methyl-2-hexenoic acid is the principal active component associated with armpit (axilla) odor, however it was not available commercially. Isovaleric (3-methylbutanoic) was chosen as the test substance, since it is closely chemically related to trans-3-methyl-2-hexenoic acid. Isovaleric acid has been used in similar research.

Isovaleric acid is associated with and contributes to foot and body perspirative malodors. Commercial samples of this malodorous material was used as a model compound to assess the ability of cosmetic compositions prepared according to the present invention, comprising synthetic metal silicate materials to remove the odors associated with these malodorous materials.

Samples for in vitro odor capacity were prepared as follows. Test specimens were prepared by weighing 0.25 grams of an odor absorbing/neutralizing test compound into a 20-ml crimp cap headspace sampling vial (VWR part no. 66064-348). Then 5 ml of 5% NaCl solution and an appropriate volume of isovaleric acid (Sigma-Aldrich part no. 3314699) was added to the vial. The volume of isovaleric acid was chosen such that the residual acid not neutralized will be within the range of the calibration curve, i.e. 20-100 μl. This volume is determined from historical data, physical properties of the test substance and trial and error. The resulting mixture was then capped, vigorously agitated on a vortex agitator, shaken by hand, allowed to equilibrate for 24 hours and then analyzed using GCMS (“Gas Chromatography Mass Spectrometry”).

The GCMS analysis took place on a system consisting of a gas chromatograph (GC) directly attached to a mass selective detector (MS). Each gaseous solute exiting the GC is ionized in an electron beam. The ions formed by a specific solute will depend on the nature of the bonds in the molecule, and both ionized molecules and ion fragments of the molecule are possible. The ions are then directed down a separator, which isolates and counts the ions according to mass. The sequence and relative intensity of the mass peaks give information about the chemical identity of the solute. The absolute intensity of the peaks provides information about the amount of substance present: the greater the amount of peak area, the greater the amount of substance that is present.

Two sampling methods were used to determine the detectable quantity of non-absorbed odor causing substance (isovaleric acid) or odor neutralization capacity of each specimen: high temperature headspace analysis and low temperature Solid Phase Microextraction (SPME) analysis.

The High Temperature Headspace Analysis was conducted using a Hewlett Packard GCMS system, which consisted of a HP 7694 headspace auto-sampler, HP 5890 Gas Chromatograph and HP 5972 Mass Selector Detector. The GC was outfitted with a Restek RTX 624 Volatiles column (30 m length, 0.25 mm id., 1.4 μm film thickness.) available from Restek Corporation, Bellfonte, Pa. The GCMS system was set to the following operating conditions.

High Temperature Odor Capacity
GCMS Operating Conditions

| | |
|---|---|
| For the Headspace: | |
| Oven temp. | 80° C. |
| Transfer/loop | 150° C. |
| Equilibration time | 15 min. |
| Loop fill time | 0.03 min. |
| Loop equilibration time | 0.05 min. |
| Loop pressurization | 0.2 min |
| time | |
| Injection time | 1.0 min. |
| For the GCMS: | |
| Temp. profile: | 5 min @ 40° C. |
| | Ramp 20° C./min to 240° C. |
| | Hold 5 min @ 240° C. |
| Injector: | 250° C. |
| | Split 100 ml/min. |
| | 4 mm straight liner with glass wool |
| Carrier gas | He, 30 cm/sec constant flow |
| Detector: | 280° C. |
| | MS scan mode 35-550 AMU |

Low temperature SPME analysis was conducted with vials, which were sampled using a 100 μm PDMS Solid Phase Microextraction fiber, available from Supelco/Sigma-Aldrich, part no. 57300-U and a manual fiber holder, part no. 57330-U. The fiber is exposed for 5 minutes at room temperature then desorbed into the GCMS. A Hewlett Packard GCMS system used for the analyses consisted of a HP 5890 Gas Chromatograph and HP 5972 Mass Selector Detector. The GC was outfitted with a Restek Stabilwax column (60 m length, 0.25 mm id., 0.25 μm df) available from Restek Corporation, Bellfonte, Pa. as part no. 10626. The GCMS system was set to the following operating conditions.

Low Temperature Odor Capacity
GCMS Operating Conditions

| For the GCMS: | |
|---|---|
| Temp. profile: | 4 min @ 50° C. |
| | Ramp 10° C./min to 200° C. |
| | Ramp 20° C./min to 240° C. |
| Carrier gas: | He, 24 psi |
| Injector: | 250° C. |
| | Split - 100 ml/min. |
| | 1 mm straight liner |
| Detector: | 280° C. |
| | MS scan mode 35-550 AMU |

A calibration curve was generated as follows. For each sampling method, 20 μl to 100 μl of isovaleric acid was added to 5 mls of 5% NaCl in sealed headspace vials. The standard vials were then analyzed as described above whether using the high temperature or low temperature procedure. Typically a linear calibration curve of R²=0.98 to 0.99 can be generated.

To compensate for day to day drift in detector response, calibration was accomplished by running replicate standards at a 60 μl loading in 5 mls 5% NaCl at the beginning and end of each analytical set. The average peak area of these runs is used to calculate a single point response factor at 60 μls.

To calculate the odor neutralizing capacity of each specimen, a known amount of isovaleric acid was added to the 0.25 g specimen in 5 mls 5% NaCl. The amount added was such that there was an excess of isovaleric acid in the vial available for headspace or SPME analysis—i.e. not neutralized. The residual amount of acid in the vial, calculated from the 60 μl response factor was subtracted from the amount added, and divided by the specimen weight. This is the amount of isovaleric acid neutralized or the odor absorbing capacity in μl/g.

$$µl Residual Isovaleric Acid = \frac{Peak Area Specimen}{60 µl \text{/} Average Peak Area 60 µl Replicates}$$

$$µl \text{/} g Isovaleric Acid Neutralized = \frac{Isovaleric Acid Added - Residual Isovaleric Acid}{Specimen Weight , g}$$

TABLE 5

| | | High | Low | |
|---|---|---|---|---|
| | | Temperature | Temperature | |
| | Composition | Odor Capacity | Odor Capacity | |
| Test material | CaO:SiO2 | (μl/g) | (μl/g) | 5% pH |
| Hubersorb 600 | 0.5:1 | 950 | 920 | 10 |
| Wollastonite | 1:1 | 300 | 305 | 9.0 |
| Example 1 | 1:1 | 1420 | — | 10.8 |
| Example 2 | 1:1 | 900 | — | 9.5 |
| Example 3 | 2:1 | 1771 | 1900 | 11.8 |
| Example 4 | 3:1 | 2110 | 2090 | 11.7 |
| Example 5 | 1:1 | 1380 | — | 9.65 |
| Example 6 | 1:1 | 1026 | — | 9.6 |
| Example 7 | 2:1 | 1740 | 1780 | 9.6 |
| Example 8 | 3:1 | 1786 | 1860 | 9.4 |

This example illustrates that synthetic amorphous calcium silicates prepared according to the present invention and having a higher mole ratio of calcium oxide to silica provide improved odor capacity. Synthetic amorphous calcium silicates with the same mole ratio, but with reduced pH essentially maintain this higher odor capacity, while being more consumer-acceptable.

### EXAMPLE 15

In this example, samples of Examples 3, 9, 10 and 11 were evaluated in an in vitro odor absorption capacity test described above. Results of the odor capacity evaluation are summarized in Table 6 below.

TABLE 6

| | | High | Low | |
|---|---|---|---|---|
| | | Temperature | Temperature | |
| | | Odor | Odor | |
| Test | | Capacity | Capacity | 5% |
| material | Composition | (μl/g) | (μl/g) | pH |
| Example 3 | 2CaO:1SiO₂ | 1770 | 1900 | 11.8 |
| Example 9 | 1MgO:1SiO₂ | 1470 | — | 9.1 |
| Example | 1CaO:1MgO:1SiO₂ | 2200 | — | 10.5 |
| 10 | | | | |
| Example | 0.5CaO:1.5MgO:1SiO₂ | 1707 | 2200 | 9.6 |
| 11 | | | | |

It is shown that all these examples had excellent odor capacity with the better capacity achieved with a 2 metal oxide:1 silica ratio. The creation of the mixed oxide is effective at reducing pH, while maintaining excellent odor capacity.

### EXAMPLE 16

In this example, the odor absorption capacity of Examples 11 inventive calcium magnesium silicate was compared to physical mixtures of calcium silicate and either magnesium silicate or magnesium carbonate.

A physical mixture of calcium silicate and magnesium silicate was made by dry blending 5.68 g calcium silicate of Example 3 with 13.5 g of magnesium silicate of Example 12 and then dry milling the mixture to homogenize the sample. The resulting blend has a molar ratio equal to Example 11 calcium magnesium silicate, but with a higher pH.

For comparative purposes, a physical mixture of 10 g Hubersorb 600 calcium silicate available from J.M. Huber Corporation, Edison, N.J. and 6.76 g magnesium carbonate (EM Science, Gibbstown, N.J.) were dry blended and milled to homogenize. This blend had the same molar ratio and pH as Example 11.

Odor capacity was evaluated in an in vitro odor absorption capacity test elsewhere. Results of the odor capacity evaluation are summarized in Table 7 below.

TABLE 7

| | | High | Low | |
|---|---|---|---|---|
| | | Temperature | Temperature | |
| | | Odor | Odor | |
| | Composition | Capacity | Capacity | |
| Test material | CaO:MgO:SiO2 | (μl/g) | (μl/g) | 5% pH |
| Example 11 | 0.5:1.5:1 | 1707 | 2200 | 9.6 |
| Blend of Example 3 and | 0.5:1.5:1 | 2079 | 1900 | 11.4 |
| Example 12 | | | | |
| Blend of Magnesium | 0.5:1.5:1 | 1000 | — | 9.6 |
| Carbonate and Hubersorb | | | | |
| 600 | | | | |

The physical blend of Example 3 and Example 12 at a composition equivalent to Example 11 gave a good odor capacity, but had a higher pH than the calcium magnesium silicate of Example 11. The comparative physical blend of Hubersorb 600 calcium silicate and magnesium carbonate had the same molar composition and pH as Example 11, but a much lower odor capacity. This example illustrates that the synthetically produced silicate as prepared according to the present invention provides synergistically improved odor absorbing capacity as compared to the comparative blend and offers the unique benefit of excellent odor capacity at an acceptable pH. Such performance would not have been expected by a person of ordinary skill in the art.

### EXAMPLE 17

The magnesium zinc silicate of Example 13 was compared to a physical blend of magnesium silicate and zinc carbonate, zinc carbonate, magnesium carbonate and magnesium aluminum silicate. The zinc carbonate and magnesium carbonate were obtained from EM Science Company, Gibbstown, N.J. The magnesium aluminum silicate used was Sebumase obtained from U.S. Cosmetics Corporation, Dayville, Conn.

The physical blend was made by combining 10 g of Example 9 magnesium silicate with 8.73 g zinc carbonate, blending and milling to homogenize.

Odor capacity was evaluated in an in vitro odor absorption capacity test described above. Results of the odor capacity evaluation are summarized in Table 8 below.

TABLE 8

| | | High Temperature | |
|---|---|---|---|
| | Composition | Odor Capacity | |
| Test material | MgO:ZnO:SiO2 | (μl/g) | 5% pH |
| Example 13 | 1:1:1 | 1300 | 9.7 |
| Example 9 and Zinc | 1:1:1 | 1500 | 8.81 |
| Carbonate blend | | | |
| Zinc Carbonate | 0:1:0 | 1073 | 8.9 |
| Magnesium Carbonate | 1:0:0 | 1165 | 9.9 |
| Magnesium Aluminum | 1:0:1 | 300 | 6.92 |
| Silicate | | | |

It is seen in Table 8 that the odor capacity of the Example 13 prepared according to the present invention and that of Example 9 prepared according to the present invention and blended with zinc carbonate exceeded the capacity of zinc or magnesium carbonate used singly and far exceeded that of the known, prior art magnesium aluminum silicate.

### EXAMPLE 18

To evaluate the odor absorbing performance and efficacy of mixed metal oxide silicates incorporated in a solid stick deodorant base product, solid stick deodorant formulations containing several of the functional silicates prepared according to the present invention and similar to that of commercial stick deodorants was produced and tested on human subjects under conditions of actual use. Various cosmetic ingredients, as listed in Table 9, below, were mixed in a glass reactor, to which a water condenser is attached, and then heated to 80-85° C. with stirring provided by a lightning mixer and Teflon agitator. Upon complete addition of all the other ingredients, the specific absorbents (in the amounts shown below) or triclosan (premixed with stearyl alcohol) was stirred in and the mixture allowed to cool to 52° C. before filling appropriate containers for clinical testing.

TABLE 9
Cyclicsilicone Solid Stick Deodorant Formulation

| | Test Article A | Test Article B | Test Article C | Test Article D |
|---|---|---|---|---|
| Description | Cyclicsilicone | Cyclicsilicone | Cyclicsilicone | Cyclicsilicone |
| | with 0.3% | with 0.5% | with 0.5% | with 0.5% |
| | triclosan | calcium | calcium | magnesium zinc |
| | | silicate | magnesium silicate | silicate of |
| | | of Example 7 | of Example 11 | Example 13 |
| Cyclomethicone | 744.3 | 744.3 | 744.3 | 744.3 |
| (SF-1202) | | | | |
| Stearyl Alcohol | 228.75 | 228.75 | 228.75 | 228.75 |
| (Universal) | | | | |
| Triclosan into stearyl | 3.13 | 0 | 0 | 0 |
| alcohol | | | | |
| Hydrogenated Castor | 30.53 | 30.53 | 30.53 | 30.53 |
| Oil (Caster Wax MP- | | | | |
| 80 Caschem) | | | | |
| Tricontanyl PVP | 7.61 | 7.61 | 7.61 | 7.61 |
| (GANEX ® WP-660 | | | | |
| ISP) | | | | |
| Phenyltrimethicone | 30.53 | 30.53 | 30.53 | 30.53 |
| (DC-556 Dow) | | | | |
| Absorbent of this | 0 | 5.21 | 5.21 | 5.21 |
| invention | | | | |

The Cyclomethicone used was SF-1202 available from GE Silicones Waterford, N.Y.; Stearyl alcohol used was Lanette 18 DEO available from Cognis Corp. Cincinnati, Ohio; Hydrogenated Castor Oil was Castor Wax MP-80 available from Frank B. Ross Co. Jersey City, N.J.; Tricontanyl PVP was GANEX® WP-660 available from Cognis Corp., Cincinnati, Ohio; and the Phenyltrimethicone was DC-556 available from Dow Coming, Taylor, Mich.

The clinical test was conducted in compliance with applicable Good Clinical Practice Regulations in accordance with Title 21 of the Code of Federal Regulations, Part 50. The study used a paired comparison test design, with 4 pairs: Example 7, Example 11 and Example 13, each paired with the antimicrobial agent triclosan. For each subject, a test article was randomly assigned to one axilla and the opposite axilla received a control test article containing triclosan. Approximately 20 subjects completed each pair. See Table 9 above for the exact compositions of the test articles.

Approximately 80 male and female subjects were selected that satisfy the following inclusion criteria consisting of age: 18 years or older and use of only a non-antimicrobial, fragrance-free soap product for all personal bathing for a minimum of 7 days prior. Subjects were free from axillary irritation, active psoriasis, eczema, skin cancer or dermatological conditions. Accepted subjects participated in a supervised wash using the soap according to the following procedure. The right axilla was washed for approximately 10 seconds using a disposable towel saturated with a 2% solution of the standard soap. A fresh disposable towel was wetted under running water and used to rinse the axilla until all soap is removed. The axilla was gently patted dry using a dry disposable towel. The procedure was repeated for left axilla.

Subjects were given a treatment assignment number corresponding to the randomization following enrollment. Each subject received two-treatment application(s) at 0.40 g±0.02 g per axilla/application. The test article was applied by a technician who will uniformly cover an approximate 4×6 cm area centered in the axillary vault. The amount of the test article used will be determined by weighing each unit before and after each use. Subjects waited for a minimum of 10 minutes prior to receiving freshly laundered white T-shirts, which they wore during the test period.

The odor was evaluated for the subjects at 10 (±0.5) hours following the second treatment application. The odor evaluation scoring system was based on an 10-point scale. The scoring system is based on a range of underarm odor from no malodor (0), to moderate malodor (5), to extremely strong malodor (10). Odor measurements were made subjectively by trained judges familiar with the procedures and rating system.

The source data were the individual subject malodor scores assigned at the post-treatment evaluation by the three trained judges. Separate analyses were conducted for each treatment pair (Test Article A verses B, Test Article A verses C and Test Article A verses D). For the paired comparison evaluation of the test articles, the three judge average differences between treatments will be analyzed using the distribution-free signed rank test as described in Non-parametric Statistical Methods, Hollander, M. and Wolfe, D. A., (1973) Chapter 3. The null hypothesis, which states that the difference between the paired test articles is equal to zero, will be rejected if the signed rank test p-value value is less than or equal to 0.05. The results are set forth in Tables 10, 11, and 12 below.

TABLE 10
Malodor Comparison of Paired Formulations

| | Test Article A | Test Article B |
|---|---|---|
| Mean Odor Score | 6.0 | 6.4 |
| % Difference | 7 | — |
| Subjects, n | 21 | 21 |

TABLE 11
Malodor Comparison of Paired Formulations

| | Test Article A | Test Article C |
|---|---|---|
| Mean Odor Score | 6.0 | 6.2 |
| % Difference | 3 | — |
| Subjects, n | 21 | 21 |

TABLE 12
Malodor Comparison of Paired Formulations

| | Test Article A | Test Article D |
|---|---|---|
| Mean Odor Score | 5.8 | 6.3 |
| % Difference | 8.6 | — |
| Subjects, n | 21 | 21 |

No significant differences in performance were observed in any pairs of materials when comparisons of malodor were made. Each of the test articles, B, C, and D that contained a silicate absorbent prepared according to the present invention but no triclosan provided clinically equivalent odor benefits.

Thus, deodorant products incorporating the silicate absorbent prepared according to the present invention provide a safe and effective alternative to sold stick deoderants incorporating triclosan.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. A fluid personal care composition comprising a metal oxide silicate capable of absorbing a malodorous compound and a vehicle; the metal oxide silicate described by the formula: x MO:SiO₂, wherein M is at least one multivalent metal cation, x is the number of moles of metal oxide, x being equal to or greater than about 1; and the metal oxide silicate has an oil absorption of greater than 50 ml/ 100 g.

2. The fluid personal care composition according to claim 1, wherein M is selected from the group consisting of calcium, magnesium, and zinc.

3. The fluid personal care composition according to claim 1, wherein x is from about 2 to about 3.

4. The fluid personal care composition according to claim 1, wherein the metal oxide silicate has an oil absorption of between about 50 ml /100 g and about 250 ml/100 g.

5. The fluid personal care composition according to claim 1, wherein the metal oxide silicate has a 5% pH of between about 9 and about 10.

6. The fluid personal care composition according to claim 1, wherein the metal oxide silicate has an average particle size of less than 30 μm.

7. The fluid personal care composition according to claim 1, wherein the at least one multivalent metal cation is selected from the group consisting of calcium and magnesium cations.

8. The fluid personal-care composition according to claim 3, wherein the at least one multivalent metal cation is selected from the group consisting of calcium and magnesium cations.

9. The fluid personal care composition according to claim 1, wherein the composition is a deodorant and is in a form selected from the group consisting of solid stick deodorants, liquid roll-on deodorants, aerosol, and pump spray deodorants, semi-solid gel deodorants, soap bars, and deodorant lotions and creams.

10. A fluid personal care composition comprising:
(a) about 0.5 wt % to about 20 wt % of a metal oxide silicate the metal oxide silicate described by the formula: x MO:SiO₂, wherein M is at least one multivalent metal cation, x is the number of moles of metal oxide, x being equal to or greater than about 1; and the metal oxide silicate has an oil absorption of greater than 50 ml/100 g; and
(b) about 80 wt % to about 99.9 wt % of other personal care composition ingredients selected from the group comprising a vehicle, thickeners, rheology modifiers, pH buffering agents, additional malodor control agents, fragrance materials, dyes, and pigments, preservatives, skin aids, cosmetic astringents, liquid or solid emollients, emulsifiers, film formers, propellants, skin-conditioning agents, such as humectants, skin protectants, solvents, solubilizing agents, suspending agents, surfactants, waterproofing agents, viscosity increasing agents, waxes, and wetting agents.

11. A method of inhibiting body odor by applying to the skin an effective amount of a personal care composition comprising a metal oxide silicate capable of absorbing a malodorous compound, the metal oxide silicate being characterized by the formula: x MO:SiO₂, wherein M is at least one multivalent metal cation, x is the number of moles of metal oxide, x being equal to or greater than about 1; and the metal oxide silicate has an oil absorption of greater than 50 ml/100 g .
